(19) United States
(12) Patent Application Publication
(10) Pub. No.: US 2003/0180279 A1
(43) Pub. Date: Sep. 25, 2003

(54) Composition and method to prevent or reduce diarrhea and steatorrhea in HIV patients

(76) Inventors: Tibor Sipos, Lebanon, NJ (US); Simantini Das, Easton, PA (US); Teresa Marie Wignot, Dallas, PA (US)

Correspondence Address: Law Office of Imre Balogh, 276 Smith School Road, Perkasie, PA, 18944, US

(21) Appl. No.: 10/100,716

(22) Filed: Mar. 19, 2002

(51) Int. Cl. A61K 38/54

(52) U.S. Cl. 424/94.2

(57) ABSTRACT

Method of preventing or reducing diarrhea and/or steatorrhea in HIV-positive patients being treated with High Activity Antiretroviral drugs containing protease inhibitors, nucleoside reverse transcriptase inhibitors or non-nucleoside reverse transcriptase inhibitors. The method includes the steps of: administering to the HIV-positive patient a High Activity Antiretroviral drug containing a protease inhibitor, a nucleoside reverse transcriptase inhibitor or a non-nucleoside reverse transcriptase inhibitor; and co-administering with the HAART drug a gastric acid-resistant polymer-coated and buffered digestive enzyme composition containing pancreatic proteases, lipases, co-lipases, nucleases, amylases and other bio-active substances produced by the pancreatic gland.

### BACKGROUND OF THE INVENTION

1. Field of the Invention

The present invention relates to a composition and method using the composition to treat and to prevent/reduce diarrhea and steatorrhea in HIV patients who are treated with High Activity Antiretroviral Therapy, hereinafter referred to as “HAART”.

2. Reported Developments

The current most effective treatment of individuals infected with Human Immunodeficiency Virus, hereinafter referred to as “HIV”, is the HAART method which comprises administering a combination of drugs that attack the HIV mechanism for viral reproduction. The therapy consists of using drugs that inhibit reverse transcriptase and HIV protease. HAART is intended to increase CD4 lymphocyte counts and suppression of HIV load in response to the antiretroviral therapy. Ultimately, the therapy results in declining HIV-related morbidity and mortality.

Drugs used in HAART include: protease inhibitors (PI); non-nucleoside reverse transcriptase inhibitors (NNRTI); and nucleoside reverse transcriptase inhibitors (NRTI). Table I lists these drugs by trade name, chemical name and type.

TABLE I
DRUGS USED IN HAART

| Trade Name | Chemical Name | Type |
|---|---|---|
| CRIXIVAN ® | Indinavir sulfate | PI |
| AGENERASE ® | Amprenavir | PI |
| NORVIR ® | Ritonavir | PI |
| FORTOVASE ® | Saquinavir | PI |
| VIRACEPT ® | Nelfinavir mesylate | PI |
| INVIRASE ® | Saquinavir mesylate | PI |
| SUSTIVA ® | Efavirenz | NNRTI |
| VIRAMUNE ® | Nevirapine | NNRTI |
| ZIAGEN ® | Abacavir sulfite | NNRTI |
| RESCRIPTA ® | Delavirdine mesylate | NNRTI |
| HIVID ® | Zalcitabine | NRTI |
| ZERIT ® | Stavudine | NRTI |
| RETROVIR ® | Zydovudine | NRTI |
| EPIVIR ® | Lambivudine | NRTI |
| COMBIVIR ® | Lambivudine, Zidovudine | NRTI |
| VIDEX ® | Didanosine | NRTI |

Using HAART, a combination of these drugs, often referred to as “cocktails”, is administered to HIV patients. The Panel on Clinical Practices convened by the Department of Health and Human Services (DHHS) and the Henry J. Kaiser Family Foundation has recently developed guidelines for the treatment of HIV. Table II shows the recommenced antiretroviral therapy for initial treatment of HIV patients.

TABLE II
RECOMMENDED ANTIRETROVIRAL THERAPY FOR
INITIAL TREATMENT OF HIV PATIENTS

| Recommendation | PI or NNRTI | NRTI |
|---|---|---|
| Strongly | Efavirenz (NNRT) | Stuvadine + Lambivudine |
| recommended | Indinavir (PI) | Stuvadine + Didanosine |
| | Nelfinavir (PI) | Zidovudine + Lambivudine |
| | Ritonavir + Saquinavir | Zidovudine + Didanosine |
| | (PI) | |
| Recommended as | Abacavir (NNRTI) | Didanosine + Lambivudine |
| Alternative | Amprenavir (PI) | |
| | Delavirdine (NNRTI) | |
| | Nelfinavir + Saquinavir | Zidovudine + Zalcitabine |
| | (PI) | |
| | Nevirapine/Ritonavir (PI) | |
| | Saquinavir (PI) | |

Many individuals infected with HIV and receiving HAART suffer from mild to severe diarrhea which is a side effect of the treatment. A recent study (Kakuda et al., “Protease Inhibitors for the treatment of human immunodeficiency virus infection”, Am. J. Health Syst. Pharm., vol. 55, no. 3, pp. 233-254, Feb. 1, 1998) states that 12-20% of HIV patients receiving nelfinavir experience diarrhea. It has also been reported that 75% of HIV-1 positive individuals that had not previously received antiretroviral therapy had episodes of diarrhea after starting a quadruple regimen of stavudine and lamivudine with nelfinavir and saquinavir (Reijers, MH et al., “Toxicity and drug exposure in a quadruple drug regimen in HIV-1 infected patients participating in the ADAM study”, AIDS, vol. 14, no. 1, pp. 59-67, Jan. 7, 2000). The detrimental effects of diarrhea include maldigestion, malabsorption of nutrients, excretion of undigested fats (steatorrhea), and unabsorbed pharmaceuticals resulting in decreased immunocompetance, and loss of muscle mass (Sherman, D S, et al., “Management of protease inhibitor-associated diarrhea”, Clin, Infect. Dis., vol., 30, no. 6, pp. 908-914, 2000).

Drug-induced diarrhea diminishes the overall therapeutic effectiveness of the HAART drugs by hindering their absorption into the patient's circulatory system. In addition, the overall quality of life of the patient is severely compromised (Watson, A., “Diarrhea and quality of life in ambulatory HIV-infected patients”, Dig. Dis. Sci., vol. 41, no. 9, pp. 1794-1800, September 1996). Due to the reduction in quality of life, compliance with drug therapy is often a serious problem. Current treatments for diarrhea of HIV-positive patients on HAART therapy include oat bran, psyllium, loperamide, calcium carbonate and other over-the-counter medications that are only partially effective. There is insufficient information on the mechanism of antiretroviral drug-induced diarrhea and steatorrhea. It is hypothesized that the combination of drugs in HAART may interfere with the production and release of the digestive components by directly inhibiting the enzyme activating cascade and/or the digestive enzymes, both lipase and proteases, or disrupt complexation of lipase with colipase or bile salts.

More than 95% of dietary fats ingested by the average adult in a day are triglycerides, and if these nutrients remain undigested, diarrhea can result. The digestion of triglycerides is chemically complicated and involves two fundamentally different but closely interrelated processes: the activation of several inactive proenzymes (zymogens), and the emulsification of nutrient lipids with bile salts. In the first part of the process, trypsin converts the zymogen procolipase, secreted by the pancreas, into the 12 kD protein colipase. In the second part of the process, colipase anchors the complex formed between a lipase and a micellar bile acid to its triglyceride substrate, thereby stabilizing the complex and activating it enzymatically; the triglyceride may now be hydrolyzed to free fatty acids and monoacyl glycerol. The trypsin needed in the first part of the process is produced by the pancreas as trypsinogen (zymogen) and activated by enterokinase, which is secreted by the Brunner's gland in the duodenum. The inhibition of pancreatic lipase or any enzyme in the zymogen-activating cascade leads to undigested fats that become hydroxylated to hydroxy fatty acids by the intestinal bacterial flora. Hydroxylated fatty acids are well-known diuretics.

### SUMMARY OF THE INVENTION

It has now been discovered that exogenous administration of a bicarbonate-buffered and enteric-coated pancrelipase to human immunodeficiency virus (HIV) positive patients who experience diarrhea due to HAART drugs which contain nucleoside reverse transcriptase inhibitors (NRTI), protease inhibitors (PI), or non-nucleoside reverse transcriptase inhibitors (NNRTI) reduces/eliminates diarrhea and/or steatorrhea.

The bicarbonate-buffered and enteric-coated pancrelipase maybe co-administered or sequentially administered with protease inhibitors (PI), nucleoside reverse transcriptase inhibitors (NRTI), or non-nucleoside reverse transcriptase inhibitors (NNRTI) to patients who are HIV-positive.

The protease inhibitors (PI), nucleoside reverse transcriptase inhibitors (NRTI), and non-nucleoside reverse transcriptase inhibitors (NNRTI) include the following drugs listed by their chemical names: Indinavir sulfate, Amprenavir, Ritonavir, Saquinavir, Nelfinavir mesylate, Saquinavir mesylate, Elfavirenz, Nevirapine, Abacavir sulfate, Delavirdine mesylate, Zalcitabine, Stavudine, Zydovudine, Lambivudine, Lambivudine/Zidovudine combo and Didanosine.

The bicarbonate-buffered and enteric-coated pancrelipase (disclosed in U.S. Pat. No. 5,578,304 and incorporated herein by reference in its entirety) preferably comprises:

(a) from about 10 to about 90% w/w of an enzyme selected from the group consisting of pancreatic proteases, lipases, co-lipase, nucleases, amylases, and bio-active substances produced by the pancreatic gland;

(b) from about 15 to 60% w/w of a buffering agent selected from the group consisting of anhydrous sodium carbonate, sodium bicarbonate, potassium carbonate, potassium bicarbonate, ammonium carbonate, tromethamine, di(tris)hydroxymethylaminomethane) carbonate, tris-glycine, di-arginine, tri-arginine, poly-arginine, di-lysine, tri-lysine, poly-lysine, diethylamine and triethanolamine, said buffering agent providing a pH of from 7 to 9 in the small intestine of a mammal, and said lipase having an activity of from 24% to 100% at said pH of from 7 to 9;

(c) from about 0.5 to about 16% w/w of a disintegrant selected from the group consisting of ursodiol, starch, modified starches, microcrystalline cellulose and propylene glycol alginate;

(d) from about 1 to about 19% w/w of an adhesive polymer selected from the group consisting of polyvinylpyrrolidone, hydroxyethyl cellulose, cellulose acetate phthalate, ethyl cellulose and hydroxypropylmethyl cellulose;

(e) from about 7.0 to about 15% w/w of a non-porous, gastric acid-resistant and pharmaceutically acceptable polymer coating which contains less than 2% talc and which is insoluble in the pH range of from about 1.5 to about 5 but is soluble in the pH range of about 5.5 to about 9, said polymer coating comprises a polymer selected from the group consisting of hydroxypropyl methyl cellulose phthalate, cellulose acetate phthalate, diethyl phthalate, dibutyl phthalate, and enteric coating polymer dispersion, and an acrylic based polymeric dispersion.

### DETAILED DESCRIPTION OF THE INVENTION

### Methods Employed

Enzyme and Drug Preparation of Lipase, Protease and Trypsin Assays

The enzyme sources consisted of USP Reference Standard Pancrelipase, USP Reference Standard Trypsin, colipase (Sigma Chemical Co.), and enterokinase (ICN Biochemicals). Appropriate concentrations of enzyme reference standard were prepared in cold distilled water, as described in USP reference assay procedures. The Infectious Disease Clinic in Somerville, N.J. provided the HAART medications. Pure drugs could not be obtained so commercial drug preparations were used in the assays. D-α-tocopheryl polyethylene glycol 1000 succinate (TPGS) was provided by Eastman Chemical Co. (Anglesey, UK); Vitamin E (HealthSmart Vitamins) was purchased from Eckerd Drugs (Bethlehem, Pa.); povidone was provided by ISP Technologies, Calvert City, Ky. Appropriate concentrations of HAART drug, drug cocktails, and excipients were prepared in distilled water and reagent-grade methanol (Aldrich, Milwaukee, Wis.) or DMSO (Aldrich, Milwaukee, Wis.).

Lipase Assay

One unit (U) of lipase activity is defined as the amount of enzyme that liberates one μequivalent of free fatty acid from triglycerides per minute at pH 9.0 and 37° C. Lipase activity (U/mg) was determined by a lipase specific titrimetric assay described in the United States Pharmacopeia (USP), Volume XXII. In brief, the substrate consists of 10% olive oil in acacia solution, 40-mg/mL sodium taurocholate, 0.075M calcium chloride, 0.05M Tris in 3.0M NaCl buffer solution (pH 7.5) and distilled water. The emulsified substrate is incubated at 37° C. and brought to pH 9.0 with 0.02N NaOH. At time zero, 1.0 mL of enzyme solution was added. A pH stat autotitrator was used to maintain pH 9.0 and the volume of 0.02 N NaOH dispensed per unit time was recorded for five (5) minutes. From the initial slope of this plot, lipase activity was calculated using the following equation:

$$U \text{/} mg = \frac{slope ( mL of NaOH dispensed \text{/} min ) \times N of NaOH \times 1000 }{mg of enzyme assayed}$$

HAART drug solutions were mixed with the substrate prior to incubation and addition of enzyme. ORLISTAT®, a known potent inhibitor of lipase was used as a positive reference control.

Recovery of Pancrelipase Activity after Inhibition by Protease Inhibitors

The above procedure was used to monitor pancrelipase activity with the exception that drug was not preincubated with the substrate. At time zero 1.0 mL of pancrelipase was added to initiate the reaction. At five (5) minutes, 1.0 mL of HAART drug solution was added to the reaction mixture to cause inhibition of lipase activity. At twelve (12) minutes an additional 1.0 mL of pancrelipase was added to the reaction mixture to overcome HAART inhibition. The reaction was monitored for twenty (20) minutes.

Pancrelipase Reactivation by Colipase

Junge et al., described the procedure for the reactivation of lipase by colipase. In brief, the above lipase assay procedure was used with the following variation: colipase solution replaced the distilled water so the concentration of colipase in the final reaction mixture was 5 μg/mL.

Trypsin Assay

One unit (U) of trypsin activity is defined as the amount of enzyme causing a change in absorbance of 0.003 per minute from a substrate under the conditions specified in the assay. Trypsin activity (U/mg) was determined by a trypsin specific spectrophotometric assay as described by Bergmeyer. In brief, 0.75 mL triethanolamine (TEA) solution (0.2 M TEA, 0.02M Ca₂Cl, pH 7.8) was mixed with 0.075 mL drug solution (drug in either DMSO or water) and after mixing, 0.025 mL of trypsin solution (0.32 mg/mL in 1.0 mM HCl) was added and this mixture was incubated at 25° C. for one minute. At time zero, 0.20 mL benzoyl-L-arginine-4-nitroanilide substrate (0.8 mM in 40% DMSO, 60% TEA solution) was added to give an assay volume of 1.05 mL. This gave final concentrations of substrate, trypsin and drug equal to 2.5 mg/mL, 7.6 μg/mL, and 2.1*10−4M respectively. The change in absorbance at 405 nm was monitored over time for five (5) minutes to check for linearity and product inhibition. Each subsequent run was determined over a one-minute span. Trypsin activity was calculated using the following equation:

$$U = \frac{ΔAbsorbance * Assay volum ( 1.05 mL ) * 1000 }{1.02 * 10 * change in time ( 1 min )}$$

HAART drug solutions were mixed with the TEA solution prior to incubation.

Enterokinase Assay

One unit (U) of enterokinase activity is defined as the amount of enzyme that will produce an increase of absorbance of 0.001 per minute at 253 nm from substrate at pH 5.6 and 25° C. The activity of enterokinase was determined by a coupled (enterokinase-trypsin) enzyme assay as provided by Biozyme. In brief, the substrate consisted of trypsinogen (1.2 mg/mL in 1.0 mM HCl/5.0 mM Ca₂Cl). A series of test tubes were labeled for each enzyme preparation and enzyme blank. Each tube contained substrate (trypsinogen) that was equilibrated to 25° C. with 0.07 M sodium succinate buffer, pH 5.6 and distilled water. To each reaction tube enterokinase solution (57.6 units/mL buffer) was added and distilled water was added to blank tubes. The total volume contributions of each component were: 1.0 mL succinate buffer, 0.8 mil distilled water, 0.1 mL of enterokinase, 0.5 mL trypsinogen, and 0.2 mL of either; drug dissolved in DMSO, drug dissolved in water, or DMSO/water as control to yield a total reaction volume of 2.6 mL. This gave reaction concentration of tryspinogen, drug, and enterokinase equal to 0.23 mg/mL, 2.2*10−4M, and 2.0 U/mL respectively. All tubes were incubated for thirty (30) minutes for the enterokinase catalyzed reaction to occur. After incubation, the reaction was quenched with 2.0 M HCl . The test and reaction solutions were added separately to N-benzoyl-L-arginine ethyl ester hydrochloride (0.25 mM in 0.067 M potassium phosphate, pH 7.6) and monitored spectrophotometrically for an increase in absorbance at 253 nm for five (5) minutes to monitor the trypsin catalyzed reaction. Units of enterokinase activity were calculated by using the following equation:

$$U \text{/} mL = \frac{Δ {A}_{253} / time ( min ) Test - Δ {A}_{253} \text{/} time ( min ) Blank \times .5 .4}{0.003 \times 0.002 \times ( P . A . ) \times .0 .024 \times 15 \times 0.100}$$

wherein

5.4=total volume (mL) of the coupled reaction

0.003=the change in A₂₅₃ per minute of trypsin as per the unit definition

0.200=volume (mL) of the enterokinase/trypsinogen solution added to the N-benzoyl-L-arginine ethyl ester hydrochloride substrate solution

P.A.=Potential Activity of trypsinogen that is a reported value found on the product label of trypsinogen

0.024=mg of trypsin per nanomole of trypsin

15=time (min) of the enterokinase/trypsinogen reaction

0.100=volume (mL) of enterokinase solution used

HAART drug solutions were mixed with the substrate prior to incubation and addition of enzyme. AGENERASE®, NORVIR®, SUSTIVA®, VIRAMUNE® and VIRACEPT® were t dissolved in DMSO. All other HAART drugs were dissolved in distilled water.

### Results

Table III contains the results for the inhibition of lipase by protease inhibitors and Table IV summarizes the IC₅₀ values. AGENERASE® (amprenavir) solution, AGENERASE® e capsules, FORTOVASE® (saquinavir), NORVIR® (ritonavir) and VIRACEPT® (nelfinavir mesylate) exhibited >30% inhibition of pancrelipase at physiological concentration. CRIXIVAN® (indinavir sulfate) exhibited no significant inhibition of pancrelipase, while INVIRASE® (saquinavir mesylate) showed non-specific inhibition. Three different INVIRASE® concentrations were tested and resulted in the same percent inhibition of pancrelipase indicative of a non-specific interaction.

TABLE III
INHIBITION OF LIPASE BY HAART DRUGS

| | | | | | |
|---|---|---|---|---|---|
| | Rec. | Aqueous | Physio. | Drug | Inhibition |
| | Dose | Solubility | Conc.* | Tested | of Lipase |
| Trade Name | (mg) | (mg/mL) | (μg/mL) | (μg/mL)** | (%) |

PROTEASE INHIBITORS (PI)

| | | | | | |
|---|---|---|---|---|---|
| AGENERASE ® | 1400 | 0.04 | 2800 | 2500 | 99 |
| Solution | | | | 500 | 99 |
| | | | | 100 | 79 |
| | | | | 50 | 33 |
| | | | | 10 | 4 |
| AGENERASE ® | 1200 | 0.04 | 2400 | 2500 | 100 |
| Capsules | | | | 500 | 74 |
| | | | | 250 | 58 |
| NORVIR ® | 600 | Insol. | | 2670 | 71 |
| | | | 1200 | 1300 | 76 |
| | | | | 670 | 36 |
| | | | | 70 | 15 |
| VIRACEPT ® | 750 | Insol. | | 3330 | 100 |
| | | | 500 | 1670 | 71 |
| | | | | 420 | 52 |
| | | | | 100 | 21 |
| FORTOVASE ® | 1200 | Insol. | | 6670 | 96 |
| | | | | 4000 | 70 |
| | | | 2400 | 2330 | 43 |
| | | | | 1330 | 32 |
| | | | | 660 | 12 |
| INVIRASE ® | 600 | 2.2 | | 1610 | 21 |
| | | | 1200 | 1290 | 24 |
| | | | | 161 | 24 |
| CRIXIVAN ® | 800 | Sol. | | 4270 | 6 |
| | | | 1600 | 1600 | 0 |

NUCLEOSIDE REVERSE TRANSCRIPTASE INHIBITORS (NRTI)

| | | | | | |
|---|---|---|---|---|---|
| COMBIVIR ®*** | 150L | 20 | | 933L | 9 |
| | 300Z | | | 2000Z | |
| | | | 300L | 397L | 15 |
| | | | 600Z | 799Z | |
| EPIVIR ® | 150 | 70 | | 1310 | 9 |
| | | | 300 | 263 | 0 |
| HIVID ® | 0.75 | 76 | 1.5 | 5 | 0 |
| RETROVIR ® | 300 | 20 | 60 | 1330 | 0 |
| VIDEX ® | 400 | 27 | | 2670 | 5 |
| | | | 800 | 800 | 1 |
| ZERIT ® | 40 | 83 | 80 | 80 | 0 |
| ZIAGEN ® | 300 | 77 | | 1320 | 7 |
| | | | 600 | 645 | 13 |
| | | | | 376 | 4 |

NUCLEOSIDE REVERSE TRANSCRIPTASE INHIBITORS
(NNRTI)

| | | | | | |
|---|---|---|---|---|---|
| RESCRIPTA ® | 400 | Insol. | | 1490 | 34 |
| | | | 800 | 896 | 18 |
| SUSTIVA ® | 600 | Insol. | | 3330 | 64 |
| | | | 1200 | 1330 | 26 |
| VIRAMUNE ® | 200 | Insol. | | 1330 | 13 |
| | | | 400 | 400 | 3 |

*Physiological concentration is calculated as follows: recommended dose (mg)/approx. volume in the duodenum (500 mL)
**Mass of drug based upon label claims: AGENERASE ® solution was used neat; AGENERASE ® capsules, INVIRASE ®, NORVIR ®, RESCRIPTA ® and VIRAMUNE ® were dissolved in 50% methanol; SUSTIVA ® was dissolved in 70% methanol; VIRACEPT ® was dissolved in 100% methanol. All NRTI were dissolved in distilled water.
***COMBIVIR ® is a mixture of the active drugs Lambivudine (L) and Zidovudine (Z).

Based on the results shown in Table III further studies were conducted on various HAART drugs. The methology and results follow hereunder.

TABLE IV
IC₅₀ VALUES FOR INHIBITORS OF PANCRELIPASE AT PH 9.0

| | IC₅₀ | IC₅₀ |
|---|---|---|
| Compound Tested | (μg/mL) | (μM) |
| AGENERASE ® solution | 89 | 176 |
| AGENERASE ® capsules | 368 | 787 |
| NORVIR ® | 381 | 528 |
| VIRACEPT ® | 443 | 780 |
| FORTOVASE ® | 2164 | 3226 |
| TPGS | 275 | 182 |
| ORLISTAT ® | 0.22 | 0.44 |

None of the NNRTI or NRTI showed significant inhibition of pancrelipase at physiological concentrations. At a concentration three times physiological level, SUSTIVA® (efavirenz) exhibited 64% inhibition of pancrelipase.

Table V contains the results of the excipients that were tested for inhibition of pancrelipase activity. The excipients contained in the drug preparations that resulted in >30% pancrelipase inhibition were tested for pancrelipase inhibition. The only excipient that resulted in significant inhibition of pancrelipase activity was TPGS, which is contained in both AGENERASE® solution and AGENERASE® capsules.

TABLE V
INHIBITION OF PANCRELIPASE AT pH 9.0 BY EXCIPIENTS

| | | | | Wt % | |
|---|---|---|---|---|---|
| | Drugs | Aqueous | Max. | Excipient in | % Inhibition |
| | Containing | Solubility | Amount in | Source Used | of |
| Excipient | Excipient | (mg/mL) | Prep (wt %) | (100% = pure) | Pancrelipase |
| PEG-400 | AGENERASE ®* | Soluble | 30 | 100 | 0 |
| | NORVIR ® | | 30 | 100 | 0 |
| Povidone | INVIRASE ® | Soluble | 25 | 40 | 17 |
| TPGS | AGENERASE ® | 200 | | 20 | 96 |
| | | | | 4 | 95 |
| | | | | 2 | 93 |
| | | | | 1 | 79 |
| | | | | 0.5 | 43 |
| | | | | 0.2 | 6 |
| | | | | 0.02 | 0 |
| Vitamin E | FORTOVASE ® | Insoluble | 0.005 | 50 | 20 |

*Both solution and capsules contain PEG-400 and TPGS

The results in Table V indicate that certain HAART drugs and/or their excipients inhibit pancrelipase.

Table VI illustrates the inhibition of pancrelipase by AGENERASE®, FORTOVASE®, NORVIRx®, VIRACEPT®, and TPGS that can be overcome by the addition of excess lipase.

TABLE VI
REACTIVATION OF DRUG INHIBITED PANCRELIPASE BY THE
ADDITION OF EXCESS LIPASE

| | % Pancrelipase | % Reactivation of |
|---|---|---|
| Drug | Activity | Pancrelipase |
| AGENERASE ® | 62 | 105 |
| FORTOVASE ® | 65 | 110 |
| NORVIR ® | 70 | 110 |
| VIRACEPT ® | 35 | 50 |
| TPGS | 55 | 98 |

Colipase Results

In order to determine the lipase inhibitory mechanism of the protease inhibitors, colipase was added to reaction mixtures that exhibited >30% inhibition of pancrelipase. Table VII illustrates that colipase had no effect on the inhibition of pancrelipase when added to reaction mixtures that contained USP lipase reference standard, NORVIR®, VIRACEPT® and ORLISTAT®. Pancrelipase inhibition was reversed, i.e. pancrelipase reactivated when colipase was added to the reaction mixtures that contained AGENERASE® (capsule and solution dosage) and TPGS. The percent of pancrelipase activity increased from 21% to 79% when excess colipase was added to a reaction mixture that contained TPGS.

TABLE VII
REACTIVATION OF DRUG INHIBITED PANCRELIPASE
BY THE ADDITION OF COLIPASE

| | | % Lipase | % Lipase |
|---|---|---|---|
| | Drug Conc. | Activity | Activity |
| Drug Tested | (mg/mL) | No Colipase | with Colipase |
| USP Reference | 0.4 | 100 | 97 |
| PANCRELIPASE | | | |
| AGENERASE ® capsules | 1.5 | 27 | 74 |
| solution | 7.5 | 20 | 63 |
| NORVIR ® | 2.5 | 14 | 15 |
| VIRACEPT ® | 5.4 | 15 | 17 |
| FORTOVASE ® | 2.3 | 15 | 26 |
| TPGS | 10.0 | 21 | 79 |
| ORLISTAT ® | 0.002 | 37 | 29 |

The inhibition of Enterokinase and trypsin by PI, NNRTI and NRTI are summarized in Tables VIII, IX and X.

As illustrated in Tables VIII, IX and X, HAART drugs did not significantly inhibit either enterokinase or trypsin activity indicating there is no interference with the zyomogen activating cascade. Therefore, the conversion of procolipase to colipase may not be effected by the HAART drugs.

TABLE VIII
INHIBITION OF ENTEROKINASE BY PROTEAE INHIBITORS (PI)

| | | | | | | Drug Tested | |
|---|---|---|---|---|---|---|---|
| | | Recommended | Aqueous | Phys. | Drug | Unit of | Inhibition of |
| | Chemical | Dose | Solubility | Conc.* | Tested | Enterokinase | Enterokinase |
| Trade Name | Name | (mg) | Mg/mL | (μg/mL) | (μg/mL**) | μg/U | (%) |
| CRIXIVAN ® | Indinavir | 800 | Soluble | 1600 | 157 | 20.1 | 0 |
| | Sulfate | | | | | | |
| AGENERASE ® | Amprenavir | 1200 | 0.04 | 2400 | 111 | 16.4 | 0 |
| Capsule | | | | | | | |
| INVIRASE ® | Saquinavir | 600 | 2.22 | 1200 | 169 | 21.7 | 0 |
| | Mesylate | | | | | | |
| VIRACEPT ® | Nelfinavir | 750 | Insoluble | 1500 | 125 | 17.8 | 0 |
| | Mesylate | | | | | | |

*Physiological concentration is calculated as follows: recommended dose (mg/approx. volume in duodenum (500 mL))
**AGENERASE ® capsule and VIRACEPT ® were dissolved in DMSO, while CRIXIVAN ® and INVIRASE ® were dissolved in distilled water

Table IX contains the result of inhibition of Trypsin by Non-Nucleoside Reverse Transcriptase Inhibitors (NNRTI).

TABLE IX
NON-NUCLEOSIDE REVERSE TRANSCRIPTASE INHIBITORS (NNRTI)

| | | | | | | Drug Tested | |
|---|---|---|---|---|---|---|---|
| | | Recommended | Aqueous | Phys. | Drug | Unit of | Inhibition of |
| | Chemical | Dose | Solubility | Conc.* | Tested | Trypsin | Trypsin |
| Trade Name | Name | (mg) | Mg/mL | (μg/mL) | (μg/mL**) | μg/U | (%) |
| RESCRIPTA ® | Delavirdine | 400 | Insoluble | 800 | | | |
| | mesylate | | | | | | |
| SUSTIVA ® | Efavirenz | 600 | Insoluble | 1200 | 66 | 649 | 0 |
| VIRAMUNE ® | Nevirapine | 200 | Insoluble | 400 | 56 | 710 | 0 |

*Physiological concentration is calculated as follows: recommended dose (mg/approx. volume in duodenum (500 mL))
**SUSTIVA ® and VIRAMUNE ® were dissolved in DMSO

Table X contains the result of inhibition of Trypsin by Nucleoside Reverse Transcriptase Inhibitors (NRTI).

TABLE X
NUCLEOSIDE REVERSE TRANSCRIPTASE INHIBITORS (NRTI)

| | | | | | | Drug Tested | |
|---|---|---|---|---|---|---|---|
| | | Recommended | Aqueous | Phys. | Drug | Unit of | Inhibition of |
| | | Dose | Solubility | Conc.* | Tested | Trypsin | Trypsin |
| Trade Name | Chemical Name | (mg) | Mg/mL | (μg/mL) | (μg/mL**) | μg/U | (%) |
| EPIVIR ® | Lambivudine | 150 | 70 | 300.0 | 48 | 763 | 0 |
| HIVID ® | Zalcitabine | 0.75 | 76 | 1.5 | | | 0 |
| | Zalcitabine | | | | 44 | 765 | 0 |
| | USP Ref. STND | | | | | | |
| RETROVIR ® | Zidovudine | 300 | 20 | 60.0 | | | 0 |
| | Zidovudine | | | | 56 | 773 | 0 |
| | USP Ref. STND | | | | | | |
| ZERIT ® | Stavudine | 40 | 83 | 80.0 | 47 | 741 | 0 |
| ZIAGEN ® | Abacavir | 300 | 77 | 600 | 141 | 654 | 0 |
| | sulfate | | | | | | |

*Physiological concentration is calculated as follows: recommended dose (mg/approx. volume in duodenum (500 mL))
**All NRTI were dissolved in distilled water

### SUMMARY

Based on these in vitro results, FORTOVASE®, NORVIR® AND VIRACEPT® inhibited pancrelipase at physiological concentrations. AGENERASE® capsule exhibited 99% inhibition of pancrelipase at physiological concentration (2800 μg/mL) and AGENERASE® solution exhibited 100% pancrelipase inhibition at physiological concentration (2400 μg/mL). TPGS also significantly inhibited pancrelipase. FORTOVASE® exhibited approximately 74% inhibition of pancrelipase at physiological concentration (2400 μg/mL). NORVIR® exhibited approximately 73% inhibition of pancrelipase at physiological concentration (1200 μg/mL). VIRACEPT® exhibited approximately 72% inhibition of pancrelipase at physiological concentration (1500 μg/mL). This inhibition can be overcome by addition of excess pancrelipase. The addition of excess colipase to reaction mixtures, inhibited by AGENERASE® formulations and TPGS, restored pancrelipase activity indicating that the TPGS and/or amprenavir interfered with the lipase/colipase interactions.

The above summarized results indicate the direct inhibition of pancrelipase by protease inhibitors that provides the scientific basis for the administration of a bicarbonate buffered and enteric-coated pancrelipase having a pH of 9.0 to treat HAART-induced diarrhea and steatorrhea.

Treatment of HIV positive patients suffering from HAART induced diarrhea have responded positively (fewer loose stools and reduced incidence of gastrointestinal distention and flatulence) to the administration of an enteric-coated bicarbonate-buffered pancrelipase delayed release capsules. Pancrelipase contains lipases, colipase, amylase, proteases, nucleases and other bioactive substances produced by the pancreatic gland. While the mechanism of action of HAART drug-induced diarrhea is not known, it is hypothesized, based on the above-described results, that the presence of greasy and oily diarrhea is indicative of interference with fat and lipid digestion by HAART drugs and by directly inhibiting pancreatic lipase in the gastrointestinal tract.

As stated in the Summary of the Invention, a gastric acid-resistant polymer-coated, buffered digestive enzyme/ursodeoxycholate composition containing from about 10% to about 90% of a concentrate of an enzyme selected from the group consisting of pancreatic proteases, pancreatic lipases, colipase, pancreatic nucleases, pancreatic amylases and other bio-active substances produced by the pancreas will reduce/eliminate diarrhea in HIV positive patients being treated with HAART drugs when co-administered or sequentially administered with said drugs.

The physicians treating HIV-positive patients under the HAART protocol will determine the frequency and amount of lipase-containing compositions necessary to counteract the diarrhea occurrence. This determination by the physician will depend on the extent of diarrhea, the cocktail of HAART drugs, and the general health of the patient. Compositions other than that disclosed in U.S. Pat. No. 5,578,304 include compositions disclosed in the following patents (all of which are incorporated herein by reference):

U.S. Pat. No. 5,460,812 discloses compositions in which there is about 10 to about 90.0% w/w of a concentrate of an enzyme selected from the group consisting of pancreatin, pancreatin proteases, pancreatic lipases, pancreatic nucleases, pancreatic amylases and other bio-active substances produced by the pancreas. The compositions include about 0.3 to about 75% of a bile salt and a buffering agent.

U.S. Pat. No. 5,324,514 discloses a composition comprising of from about 71 to 90% w/w of a concentrate of an enzyme selected from the group consisting of pancreatic proteases, lipases, nucleases, and amylases; of from about 0.3% to about 13% w/w of a bile salt; and of from about 0.8% to about 5% w/w of a buffering agent.

U.S. Pat. No. 5,260,074 discloses a digestive enzyme and bile salt composition comprising:

of from about 71 to about 90% w/w of an enzyme selected from the group consisting of pancreatic proteases, lipases, nucleases and amylases;

about 1.0 to about 61% w/w of a salt of ursodeoxycholic acid selected from the group consisting of sodium, potassium, ammonium, tromethamine, ethanolamine, diethanolamine, and triethanolamine;

about 0.8 to about 5.0% w/w of a buffering agent;

about 0.3 to about 19% w/w of an adhesive polymer selected from the group consisting of hydroxypropyl cellulose, polyvinylpyrrolidone, cellulose acetate, phthalate and methyl cellulose;

about 0.9 to about 16% w/w of a disintegrant selected from the group consisting of starch, modified starches, microcrystalline cellulose and propylene glycol alginate; and

a gastric acid-resistant polymer coating the listed ingredients which disintegrates under neutral or basic conditions.

U.S. Pat. Nos. 5,578,304, 5,460,812, 5,324,514 and 5,260,074 relate to gastric acid-resistant compositions in which the coatings on the compositions do not release the active ingredients in the acidic pH of the stomach, but dissolve in the neutral or slightly basic environment of the intestines in which the active ingredients are then released.

The compositions and methods disclosed in the aforementioned patents are directed to the treatment of digestive disorders, pancreatic enzyme insufficiency, impaired liver function, cystic fibrosis, regulating the absorption of dietary iron and cholesterol and for dissolving gallstones. None of these patents suggest the use of these compositions to reduce and/or eliminate diarrhea/steatorrhea in HIV-positive patients treated with HAART. The compositions do, however, contain lipase and co-lipase and other bio-active substances produced by the pancreas which, as shown above, helps to reduce and/or eliminate diarrhea/steatorrhea in HIV-positive patients.

Other compositions containing pancreatin coated with gastric juice-resistant polymers may also be used (see for example, U.S. Pat. Nos. 4,280,971 and 5,378,462, which are incorporated herein by reference).

Various modifications of the present invention disclosed will become apparent. This invention is intended to include such modifications to be limited only by the scope of the claims.

What is claimed is:

1. A method of preventing or reducing diarrhea and/or steatorrhea in an HIV-positive patient associated with the treatment of with High Activity Antiretroviral drugs which comprise of protease inhibitors, nucleoside reverse transcriptase inhibitors, non-nucleoside reverse transcriptase inhibitors or a combination thereof, comprising the steps of:
a) administering to said HIV-positive patient a drug comprising a protease inhibitor, a nucleoside reverse transcriptase inhibitor, a non-nucleoside reverse transcriptase inhibitor, or a combination thereof contained in a pharmaceutically acceptable vehicle;
b) administering simultaneously or subsequently to said High Activity Antiretroviral drugs, a buffered and enteric-coated composition comprising:
of from about 10 to about 90% of an enzyme selected from the group consisting of pancreatic proteases, lipases, co-lipases, nucleases, amylases and other bio-active substances produced by the pancreatic gland;
of from about 15 to about 60% of a buffering agent selected from the group consisting of: anhydrous sodium carbonate, sodium bicarbonate, potassium carbonate, potassium bicarbonate, ammonium carbonate, tromethamine, di(tris)hydroxymethyl-aminomethane carbonate, tris-glycine, di-arginine, tri-arginine, poly-arginine, di-lysine, tri-lysine, poly-lysine, diethylamine and triethanolamine, said buffering agent providing a pH of from 7 to 9 in the small intestine of a patient, and said lipase having an activity of from about 24% to about 100% at said pH of from 7 to 9;
of from about 0.5 to about 16% w/w of a disintegrant selected from the group consisting of ursodiol, starch, modified starches, microcrystalline cellulose and propylene glycol alginate;
of from about 1 to about 19% w/w of an adhesive polymer selected from the group consisting of polyvinylpyrrolidone, hydroxyethyl cellulose, cellulose acetate phthalate, ethyl cellulose and hydroxypropylmethyl cellulose;
of from about 7 to about 15% w/w of a non-porous, gastric acid-resistant and pharmaceutically acceptable polymer coating which contains less than 2% talc and which is insoluble in the pH range of from about 1.5 to about 5 but is soluble in the pH range of about 5.5 to about 9, said polymer coating comprises a polymer selected from the group consisting of hydroxypropyl methyl cellulose phthalate, cellulose acetate phthalate, diethyl phthalate, dibutyl phthalate, enteric coating polymer dispersion, and an acrylic based polymeric dispersion.

2. The method of claim 1 wherein said protease inhibitor is selected from the group consisting of: indinavir sulfate, amprenavir, ritonavir, saquinavir, nelfinavir mesylate, and saquinavir mesylate.

3. The method of claim 1 wherein said nucleoside reverse transcriptase inhibitor is selected form the group consisting of: zalcitabine, stavudine, zydovudine, lambivudine, lambivudine/zidovudine combo and didanosine.

4. The method of claim 1 wherein said non-nucleoside reverse transcriptase inhibitor is selected from the group consisting of: efavirenz, nevirapine, abacavir sulfate, and delavirdine mesylate.

5. The method of claim 1 wherein said bicarbonate-buffered and enteric-coated compositions comprising of from about 10 to 90% of an enzyme selected from the group consisting of pancreatic proteases, lipases, co-lipases, nucleases, amylases and other bio-active substances produced by the pancreatic gland.

6. The method of claim 1 wherein said co-enzyme is a co-lipase.

7. A composition for preventing or reducing diarrhea and/or steatorrhea in HIV-positive patients treated with High Activity Antiretroviral drugs comprising:
a protease inhibitor, a nucleoside reverse transcriptase inhibitor, a non-nucleoside reverse transcriptase inhibitor, or a combination thereof contained in a pharmaceutically acceptable vehicle;
a buffered and enteric-coated composition comprising:
of from about 10 to about 90% of an enzyme selected from the group consisting of pancreatic proteases, lipases, co-lipases, nucleases, amylases and other bio-active substances produced by the pancreatic gland;
of from about 15 to about 60% of a buffering agent selected from the group consisting of: anhydrous sodium carbonate, sodium bicarbonate, potassium carbonate, potassium bicarbonate, ammonium carbonate, tromethamine, di(tris)hydroxymethyl-aminomethane carbonate, tris-glycine, di-arginine, tri-arginine, poly-arginine, di-lysine, tri-lysine, poly-lysine, diethylamine and triethanolamine, said buffering agent providing a pH of from 7 to 9 in the small intestine of a patient, and said lipase having an activity of from about 24% to about 100% at said pH of from 7 to 9;
of from about 0.5 to about 16% w/w of a disintegrant selected from the group consisting of ursodiol, starch, modified starches, microcrystalline cellulose and propylene glycol alginate;
of from about 1 to about 19% w/w of an adhesive polymer selected from the group consisting of polyvinylpyrrolidone, hydroxyethyl cellulose, cellulose acetate phthalate, ethyl cellulose and hydroxypropylmethyl cellulose;
of from about 7 to about 15% w/w of a non-porous, gastric acid-resistant and pharmaceutically acceptable polymer coating which contains less than 2% talc and which is insoluble in the pH range of from about 1.5 to about 5 but is soluble in the pH range of about 5.5 to about 9, said polymer coating comprises a polymer selected from the group consisting of hydroxypropyl methyl cellulose phthalate, cellulose acetate phthalate, diethyl phthalate, dibutyl phthalate, enteric coating polymer dispersion, and an acrylic based polymeric dispersion.

8. A composition for preventing or reducing diarrhea and/or steatorrhea in HIV-positive patients treated with High Activity Antiretroviral drugs comprising:
a protease inhibitor, a nucleoside reverse transcriptase inhibitor, a non-nucleoside reverse transcriptase inhibitor, or a combination thereof contained in a pharmaceutically acceptable vehicle;
a buffered and enteric-coated composition comprising:
of from about 10 to about 90% of co-lipase produced by the pancreatic gland;
of from about 15 to about 60% of a buffering agent selected from the group consisting of: anhydrous sodium carbonate, sodium bicarbonate, potassium carbonate, potassium bicarbonate, ammonium carbonate, tromethamine, di(tris)hydroxymethyl-aminomethane carbonate, tris-glycine, di-arginine, tri-arginine, poly-arginine, di-lysine, tri-lysine, poly-lysine, diethylamine and triethanolamine, said buffering agent providing a pH of from 7 to 9 in the small intestine of a patient;
of from about 0.5 to about 16% w/w of a disintegrant selected from the group consisting of ursodiol, starch, modified starches, microcrystalline cellulose and propylene glycol alginate;
of from about 1 to about 19% w/w of an adhesive polymer selected from the group consisting of polyvinylpyrrolidone, hydroxyethyl cellulose, cellulose acetate phthalate, ethyl cellulose and hydroxypropylmethyl cellulose;
of from about 7 to about 15% w/w of a non-porous, gastric acid-resistant and pharmaceutically acceptable polymer coating which contains less than 2% talc and which is insoluble in the pH range of from about 1.5 to about 5 but is soluble in the pH range of about 5.5 to about 9, said polymer coating comprises a polymer selected from the group consisting of hydroxypropyl methyl cellulose phthalate, cellulose acetate phthalate, diethyl phthalate, dibutyl phthalate, enteric coating polymer dispersion, and an acrylic based polymeric dispersion.
